(19) United States
(12) Patent Application Publication
(10) Pub. No.: US 2020/0405934 A1
(43) Pub. Date: Dec. 31, 2020

(54) DIALYSIS SYSTEM FOR FACILITATING A HEALTH-ENHANCED HOME ENVIRONMENT

(71) Applicant: Fresenius Medical Care Holdings, Inc., Waltham, MA (US)

(72) Inventors: David Yuds, Hudson, NH (US); Samiullah K. Durrani, Harvard, MA (US)

(21) Appl. No.: 16/452,680

(22) Filed: Jun. 26, 2019

(51) Int. Cl. A61M 1/14; C02F 1/68; C02F 1/00; B01D 46/00; B01D 53/26; G16H 40/63; B67D 1/00

(52) U.S. Cl. CPC A61M 1/14; C02F 1/685; C02F 1/001; B01D 46/0027; F24F 6/00; G16H 40/63; B67D 1/0003; C02F 2209/008; A61M 2205/05; B01D 53/26

(57) ABSTRACT

A dialysis system is described having components for facilitating a health-enhanced environment in the vicinity of a dialysis machine in the home. The health-enhancing components may include air improvement components and potable water supply components integrated and/or otherwise incorporated into the home dialysis machine. By incorporating the ability for monitoring, cleaning, and controlling air and/or water quality into the home dialysis machine, the dialysis machine is no longer just a source of life for the dialysis patient but also a source of health enhancement for the entire household. The system may further display and/or remotely communicate information concerning the dialysis treatment, the air quality and the potable water quality corresponding to operation of the dialysis machine in the home environment.

### TECHNICAL FIELD

This application relates generally to systems and techniques concerning a dialysis system for facilitating a health-enhanced home environment.

### BACKGROUND

Dialysis is a treatment used to support a patient with insufficient renal function. The two principal dialysis methods are hemodialysis (HD) and peritoneal dialysis (PD). During hemodialysis, the patient's blood is removed, e.g. via an arteriovenous (AV) fistula or other methods (e.g., AV graft), and passed through a dialyzer of a dialysis machine while also passing a dialysis solution, referred to as dialysate, through the dialyzer. A semi-permeable membrane in the dialyzer separates the blood from the dialysate within the dialyzer and facilitates the exchange of waste products (e.g., urea, creatine, potassium, etc.) between the blood stream and the dialysate. The membrane prevents the transfer of blood cells, protein, and other important components in the blood stream from entering the dialysate solution. The cleaned blood stream is then returned to the patient's body. In this way, the dialysis machine functions as an artificial kidney for cleaning the blood in patients with insufficient renal function.

During peritoneal dialysis, the patient's peritoneal cavity is periodically infused with dialysate. The membranous lining of the patient's peritoneum acts as a natural semi-permeable membrane that allows diffusion and osmosis exchanges to take place between the solution and the blood stream. Automated PD machines, sometimes referred to as PD cyclers, are designed to control the PD treatment process so that it can be performed at home without clinical staff, typically while the patient sleeps overnight. The process is referred to as continuous cycler-assisted peritoneal dialysis (CCPD). Many PD cyclers are designed to automatically infuse, dwell, and drain dialysate to and from the peritoneal cavity. The PD treatment typically lasts several hours, often beginning with an initial drain phase to empty the peritoneal cavity of used or spent dialysate that was left in the peritoneal cavity at the end of the last PD treatment. The sequence then proceeds through a progression of fill, dwell, and drain phases that follow sequentially. A group of fill, dwell, and drain phases, in order, can be referred to as a PD cycle.

Both PD and HD treatments may be performed at home using appropriate equipment. A chronic kidney disease patient who elects to perform home dialysis has the benefits of convenience and increase treatment frequency over receiving dialysis treatments in a dialysis clinic. However, the home environment presents other challenges, such as maintaining a clean environment, like that found in a dialysis clinic. The International Society for Hemodialysis lists the guidelines for home dialysis environments that include a low humidity and comfortable temperature.

Accordingly, it would be desirable to provide a system that helps facilitate a home environment suitable for dialysis treatments and general health.

### SUMMARY

According to an implementation of the system described herein, a dialysis system includes a dialysis machine and at least one health-enhancing component integrated into the dialysis machine. The at least one health-enhancing component includes one or more of: an air improvement component that monitors and/or treats air in a vicinity of the dialysis machine, or a potable water supply component that monitors and/or provides potable water from the dialysis machine. The air improvement component may include one or more of the following: an air purifier, a dehumidifier, an air conditioner, or an air heater. The potable water supply component may include one or more of the following: a water purifier, a minerals supply connection, or a potable water access outlet. The potable water supply component may also include a minerals supply cartridge coupled to the minerals supply connection. The at least one health-enhancing component may include a sensor, and the dialysis system may include at least one processor that receives data from the sensor and generates a report concerning the data. The sensor may include one or more of an air monitor sensor or a water monitor sensor. The dialysis system may further include a remote computer that receives the report over a network from the at least one processor and/or transmits information corresponding to the at least one health-enhancing component to the at least one processor at the dialysis machine.

According to another implementation described herein, a method for providing a health-enhanced home environment includes providing a dialysis machine that is capable of delivering a dialysis treatment. The method further includes activating at least one health-enhancing component that is integrated into the dialysis machine. The at least one health-enhancing component includes one or more of: an air improvement component that monitors and/or provides treated air in a vicinity of the dialysis machine, or a potable water supply component that monitors and/or provides potable water from the dialysis machine. The air improvement component may include one or more of the following: an air purifier, a dehumidifier, an air conditioner, or an air heater. The potable water supply component may include one or more of the following: a water purifier, a minerals supply connection, or a potable water access outlet. The potable water supply component may also include a minerals supply cartridge coupled to the minerals supply connection. The at least one health-enhancing component may include a sensor, and the method may further comprise receiving, by at least one processor of the dialysis machine, data from the sensor, and generating a report concerning the data. The sensor may include one or more of an air monitor sensor or a water monitor sensor. The method may further comprise transmitting, to a remote computer, the report over a network from the at least one processor and/or receiving, from the remote computer, information corresponding to the at least one health-enhancing component.

### BRIEF DESCRIPTION OF THE DRAWINGS

Embodiments and features of the system described herein are explained with reference to the several figures of the drawings, which are briefly described as follows.

FIG. 1 shows a dialysis system according to an example implementation of the system described herein.

FIG. 2 is a block diagram of an example computer system illustrated in connection with the dialysis machine.

FIG. 3 is a schematic illustration showing incorporation of air improvement components into a home dialysis machine according to an implementation of the system described herein.

FIG. 4 is a schematic illustration showing incorporation of potable water supply components into a home dialysis machine according to an implementation of the system described herein.

FIG. 5 is a schematic illustration of a dialysis system which is shown in connection with assessing, displaying and/or transmitting reports on air and water quality measured according to the system described herein.

FIG. 6 is a schematic illustration of a home dialysis environment according to an implementation of the system described herein.

FIG. 7 is a schematic illustration of a connected health network exchanging information with multiple home dialysis environments according to an implementation of the system described herein.

FIG. 8 is a flow diagram of operations of for facilitating a health-enhanced home environment using a dialysis machine according to the system described herein.

### DETAILED DESCRIPTION

In various embodiments and implementations, a dialysis system is described having components for facilitating a health-enhanced environment in the vicinity of a dialysis machine in the home. The health-enhancing components may include air improvement components and potable water supply components integrated and/or otherwise incorporated into the home dialysis machine. By incorporating the ability for monitoring, cleaning, and controlling air and/or water quality into the home dialysis machine, the dialysis machine is no longer just a source of life for the dialysis patient but also a source of health enhancement for the entire household.

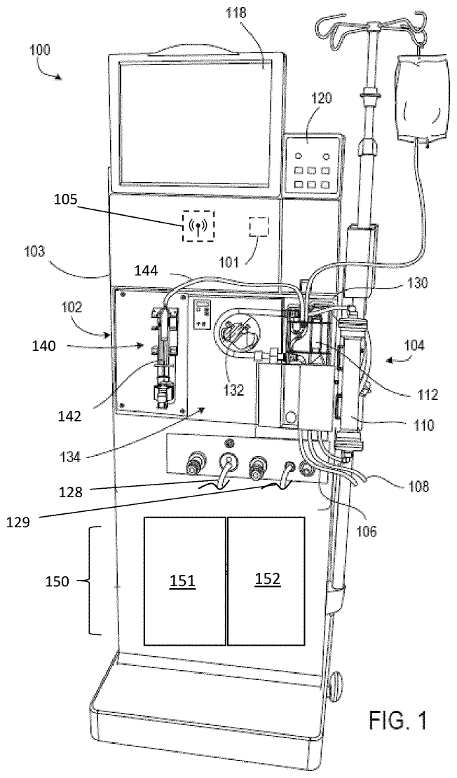

FIG. 1 shows a dialysis system 100 according to an implementation of the system described herein. The dialysis system 100 includes dialysis machine 102, such as a home hemodialysis machine, connected to a disposable blood component set 104 that partially forms a blood circuit. During hemodialysis treatment, an operator connects arterial and venous patient lines 106, 108 of the blood component set 104 to a patient. The blood component set 104 may include an air management device 112 that may include, for example, a venous drip chamber. The dialysis machine 102 includes a user interface with input devices such as a touch screen 118 and a control panel 120. The touch screen 118 and the control panel 120 allow the operator to input various different treatment parameters to the dialysis machine 102 and to otherwise control the dialysis machine 102. The touch screen 118 displays information to the operator of the dialysis system 100. Although principally discussed herein in connection with a particular configuration of hemodialysis machine, the system described herein may be used and implemented in connection with other configurations or types of hemodialysis machines, as well as other medical devices, including peritoneal dialysis machines, as will be appreciated by one skilled in the art.

The dialysis machine 102 also includes a control unit 101 (e.g., a processor) configured to receive signals from and transmit signals to the touch screen 118 and the control panel 120. The control unit 101 can control the operating parameters of the dialysis machine 102, for example, based at least in part on the signals received by the touch screen 118 and the control panel 120. The dialysis machine 102 may also include a communication unit 105 that may be provided for wireless communication with a remote control device, remote server and/or network and may be communicatively coupled with the control unit 101.

The blood component set 104 is secured to a module 130 attached to the front of the dialysis machine 102. The module 130 includes a blood pump 132 capable of circulating blood through the blood circuit. The module 130 also includes various other instruments capable of monitoring the blood flowing through the blood circuit. The blood pump 132 is part of a blood pump module 134. The blood pump module 134 includes a display window and control keys, such as a start/stop key, an up key, a down key, and a level adjust key, and an arterial pressure port. The display window displays the blood flow rate setting during blood pump operation. The start/stop key starts and stops the blood pump 132. The up and down keys increase and decrease the speed of the blood pump 132. The level adjust key raises a level of fluid in a drip chamber.

The dialysis machine 102 further includes a dialysate circuit formed by the dialyzer 110, various other dialysate components, and dialysate lines connected to the dialysis machine 102. Many of these dialysate components and dialysate lines are inside the housing 103 of the dialysis machine 102 and are thus not visible in FIG. 1. During treatment, while the blood pump 132 circulates blood through the blood circuit, dialysate pumps (not shown) circulate dialysate through the dialysate circuit.

A drain line 128 and an ultrafiltration line 129 extend from the dialysis machine 102. The drain line 128 and the ultrafiltration line 129 are fluidly connected to the various dialysate components and dialysate lines inside the housing 103 of the dialysis machine 102 that form part of the dialysate circuit. During hemodialysis, the dialysate supply line carries fresh dialysate to the portion of the dialysate circuit located inside the dialysis machine 102. As noted above, the fresh dialysate is circulated through various dialysate lines and dialysate components, including the dialyzer 110, that form the dialysate circuit. As the dialysate passes through the dialyzer 110, it collects toxins from the patient's blood. The resulting spent dialysate is carried from the dialysate circuit to a drain via the drain line 128. When ultrafiltration is performed during treatment, a combination of spent dialysate (described below) and excess fluid drawn from the patient is carried to the drain via the ultrafiltration line 129.

The dialyzer 110 serves as a filter for the patient's blood. The dialysate passes through the dialyzer 110 along with the blood, as described above. A semi-permeable structure (e.g., a semi-permeable membrane and/or semi-permeable microtubes) within the dialyzer 110 separates blood and dialysate passing through the dialyzer 110. This arrangement allows the dialysate to collect toxins from the patient's blood. The filtered blood exiting the dialyzer 110 is returned to the patient. The dialysate exiting the dialyzer 110 includes toxins removed from the blood and is commonly referred to as “spent dialysate.” The spent dialysate is routed from the dialyzer 110 to a drain.

A drug pump 140 also extends from the front of the dialysis machine 102. The drug pump 140 is a syringe pump that includes a clamping mechanism configured to retain a syringe 142 of the blood component set 104. The drug pump 140 also includes a stepper motor configured to move the plunger of the syringe 142 along the axis of the syringe 142. A shaft of the stepper motor is secured to the plunger in a manner such that when the stepper motor is operated in a first direction, the shaft forces the plunger into the syringe, and when operated in a second direction, the shaft pulls the plunger out of the syringe 142. The drug pump 140 can thus be used to inject a liquid drug (e.g., heparin) from the syringe 142 into the blood circuit via a drug delivery line 124 during use, or to draw liquid from the blood circuit into the syringe 142 via the drug delivery line 124 during use.

The dialysis machine 100 may further include home health-enhancing components 150 for facilitating a health-enhanced home environment that may be integrated into the dialysis machine 102 that, in one or more embodiments, may be a dialysis machine configured for use in the home. As further discussed in detail elsewhere herein, the home environment health-enhancing components 150 may include air improvement components 151, such as an air purifier, a dehumidifier, an air conditioner, an air heater and/or air monitor sensor(s), integrated or otherwise incorporated into the dialysis machine 102, and/or potable water supply components 152, such as a water filtration and purifier device, a mineral supplier component, a water tap, line or other access port and/or water monitor sensor(s).

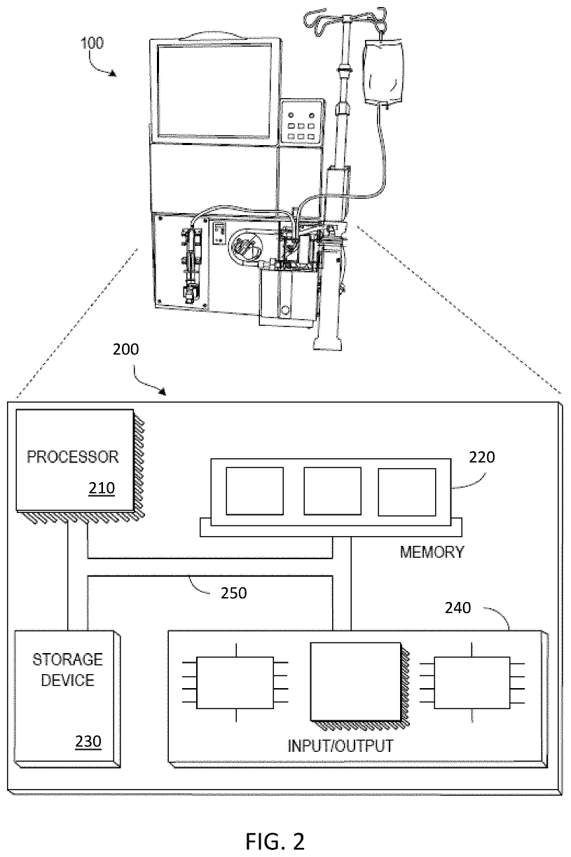

FIG. 2 is a block diagram of an example computer system 200 illustrated in connection with the dialysis machine 100. For example, the control unit 101 discussed above could be an example of the system 200 described here. The system 200 includes a processor 210, a memory 220, a storage device 230, and an input/output device 240. Each of the components 210, 220, 230, and 240 can be interconnected, for example, using a system bus 250. The processor 210 is capable of processing instructions for execution within the system 200. The processor 210 can be a single-threaded processor, a multi-threaded processor, and/or other computer. The processor 210 is capable of processing instructions stored in the memory 220 or on the storage device 230. The memory 220 stores information within the system 200. In some implementations, the memory 220 is a computer-readable medium. The memory 220 can, for example, be a volatile memory unit or a non-volatile memory unit.

The storage device 230 is capable of providing mass storage for the system 200. In some implementations, the storage device 230 is a non-transitory computer-readable medium. The storage device 230 can include, for example, a hard disk device, an optical disk device, a solid-state drive, a flash drive, magnetic tape, or some other large capacity storage device. The storage device 230 may alternatively be a cloud storage device, e.g., a logical storage device including multiple physical storage devices distributed on a network and accessed using a network. In some implementations, the information stored on the memory 220 can also or instead be stored on the storage device 230.

The input/output device 240 provides input/output operations for the system 200. In some implementations, the input/output device 240 includes one or more of network interface devices (e.g., an Ethernet card), a serial communication device (e.g., an RS-232 10 port), and/or a wireless interface device (e.g., a short-range wireless communication device, an 802.11 card, a wireless modem (e.g. 3G, 4G, 5G)). In some implementations, the input/output device 240 includes driver devices configured to receive input data and send output data to other input/output devices, e.g., a keyboard, a printer, and display devices (such as the touch screen display 118). In some implementations, mobile computing devices, mobile communication devices, and other devices are used. The input/output device 240 may further include the communication component 105 that is discussed in more detail elsewhere herein.

In some implementations, the system 200 is a microcontroller. A microcontroller is a device that contains multiple elements of a computer system in a single electronics package. For example, the single electronics package could contain the processor 210, the memory 220, the storage device 230, and input/output devices 240.

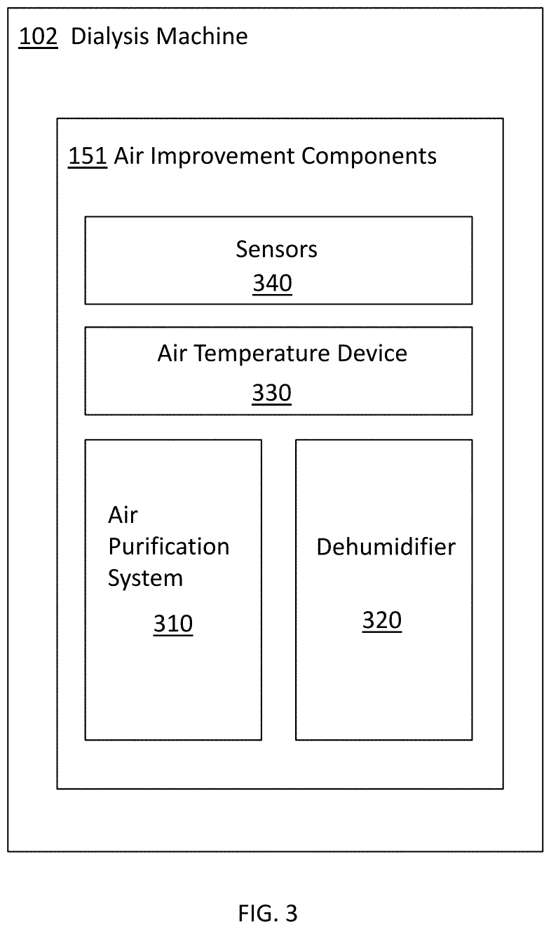

FIG. 3 is a schematic illustration showing incorporation of air improvement components 151 into a home dialysis machine, such as the dialysis machine 102. In an implementation, an air purification system 310, such as a hospital grade air purifier, may be integrated into the dialysis machine 102. The air purification system 310 may use an existing fan in the dialysis machine 102 or may be a larger fan dedicated to purifying the air. In addition to cooling the electronics, the system's fan may draw in the air in the treatment area, filter it, and circulate it to remove fine particulate pollution, pet dander, smoke, mold spores, bacteria, dust, and pollen.

Since the humidity of the home environment is also desirably maintained within certain bounds, the air improving components of the system described herein may include a dehumidifier 320 that collects excess moisture from the treatment area air and drains it through the existing dialysis waste drain lines. The condensing coils of the dehumidifier 320 may also be employed to provide cooler air for the patient if the temperature of the room is too high. In another implementation, the air improving components may further include a separate air temperature device 330, such as an air conditioner to provide cooler air and/or an air heater to provide hotter air. It is noted that clinics typically keep their temperatures at around 72° F. to inhibit bacterial growth, so the heater may be set to generally not increase the temperature beyond that point even though the patients may oftentimes feel cold at that temperature.

One or more sensors 340 may also be provided to monitor and provide measurements and indications of the air quality. For example, the sensors 340 may indicate when it is time to replace the filter or clean the system. New filters may be part of the regular home dialysis supply deliveries. The home dialysis machine 102 may also display the efficacy of the air purification to let the patient know how much cleaner the air is. By linking to online air quality resources, such as via connection to a network, the home dialysis machine 102 may also provide tips to maintain that healthier environment, like reminders to close windows when the outdoor air quality worsens. The home dialysis machine may even send reports to the patient's clinic about the air quality and such data may be used to correlate to the patient's general health, like the patient's oxygen saturation reading, also obtainable by equipment that may be linked to the dialysis machine 102, when it is time for the patient's treatment, the dialysis machine 102 could turn on a fan that blows hospital-grade air into a specific zone that is indicated on the display screen to clear away contaminants from an area that needs to be as sanitary as possible. The sensors 340 may further include other air monitoring sensors, such as air temperature and humidity sensors, as well as carbon monoxide detectors that may generate an alarm or alert in response to detection of elevated carbon monoxide level spike, for example, due to some malfunction in the patient's home.

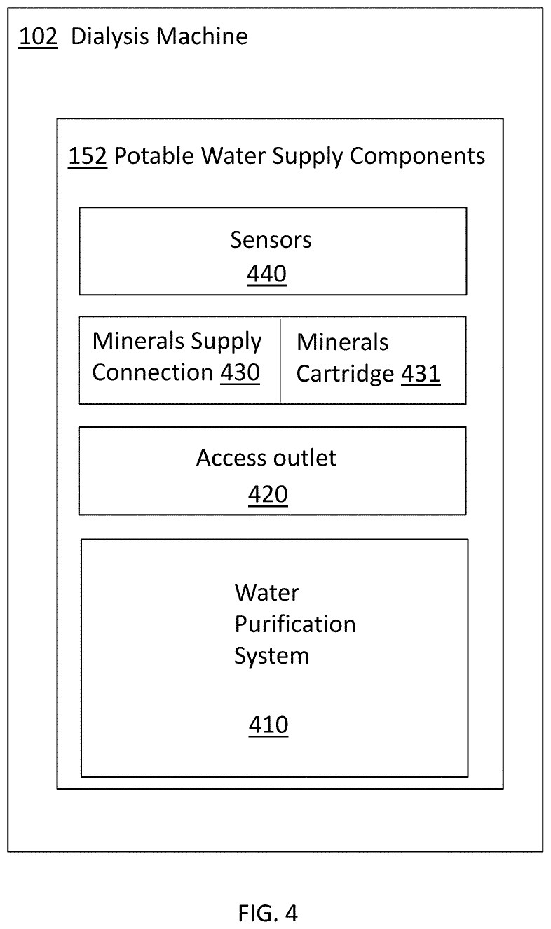

FIG. 4 is a schematic illustration showing incorporation of potable water supply components 152 into a home dialysis machine, such as the dialysis machine 102. The home dialysis machine 102 may include a water purification system 410 to produce ultra-pure water for each three hour dialysis treatment, but the equipment may be otherwise unused for treatments. The water purification system 410 may include a water filtration system using filters and/or may include another type of water purification system, such as an UV purification system. A tap, line and/or other access outlet or port 420 may be included for the ultra-pure water from the home dialysis system to provide potable water to the entire household. This may be especially beneficial where clean, potable water is not readily available. Ultra-pure dialysis quality water may lack minerals like calcium, magnesium, manganese, and iron, which are essential for health. Accordingly, the system described herein may include a minerals supply inlet or connection 430, such as a connection allowing insertion of a minerals supply cartridge 431, to add these essential minerals to the potable water. New mineral supply cartridges may be part of the regular home dialysis supply deliveries. One or more sensors 440 may also be provided to monitor and provide measurements and indications of the water quality. For example, the sensors 440 may indicate when it is time to replace the clean or replace a water filter in the water purification system 410. New filters may be part of the regular home dialysis supply deliveries. By providing these potable water supply components, the dialysis machine 102 would provide a stream of healthy, potable water for the entire household, which would be particularly beneficial where there is no other readily available alternative for clean, potable water. In an implementation, the water may be provided at various temperatures settable by the user: cold (cooled by the condenser coils), inlet temperature, and hot (heated by the dialysis machine dialysate heating element).

Combining the water purification system with the home dialysis system may simplify the setup and maintenance of the equipment. As further discussed elsewhere herein, the system may further provide detailed reports about the output water quality and filter efficacy and which may be displayed on the screen of the dialysis machine and/or, using one or more of the components of the computer system 200, may be transmitted via a network to a remote computer, database and/or server, including via a cloud network or service, for analysis and/or in connection with transmission of treatment reports concerning the dialysis treatments.

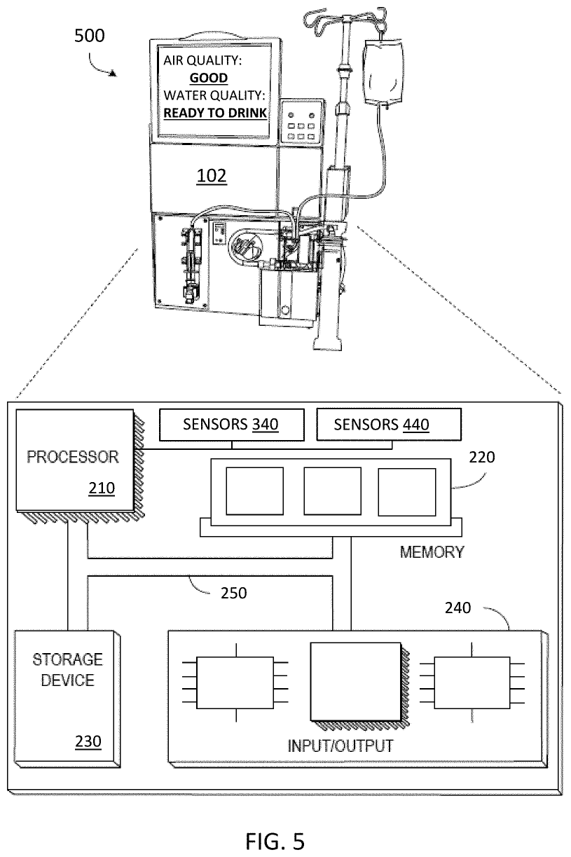

FIG. 5 is a schematic illustration of a dialysis system 500, like the dialysis system 100, which is shown in connection with assessing, displaying and/or transmitting reports on air and water quality measured according to the system described herein. In an implementation, the system 500 may further provide detailed reports about the output air quality and filter efficacy, using data from the sensors 340, and which may be displayed on the screen of the dialysis machine and/or, using one or more of the components of the computer system 200, may be transmitted via a network to a remote computer, database and/or server, including via a cloud network or service, for analysis and/or in connection with transmission of treatment reports concerning the dialysis treatments. For example, as illustrated, the dialysis machine 102 may display on screen that that Air Quality in the vicinity of the dialysis machine 102 is “Good.” Similarly, this information may also be transmitted remotely, for example, via a report.

Additionally and/or alternatively, in another implementation, the system 500 may provide detailed reports about the output water quality and filter efficacy, using data from the sensors 440, and which may be displayed on the screen of the dialysis machine 102 and/or, using one or more of the components of the computer system 200, may be transmitted via a network to a remote computer, database and/or server, including via a cloud network or service, for analysis and/or in connection with transmission of treatment reports concerning the dialysis treatments. For example, as illustrated, the dialysis machine 102 may display on screen that that Water Quality accessible at the dialysis machine 102 is “Ready to Drink.” Similarly, this information may also be transmitted remotely, for example, via a report.

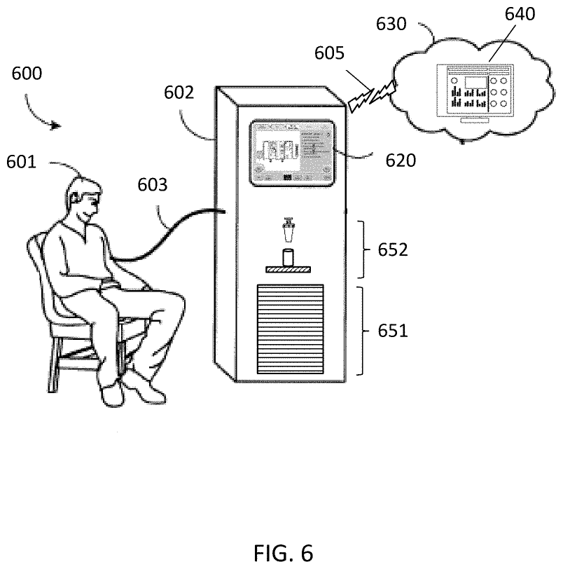

FIG. 6 is a schematic illustration of a home dialysis environment 600 according to an implementation of the system described herein. As illustrated, in the home dialysis environment 600, a patient 601 is receiving a dialysis treatment, via tubing 603, from a dialysis machine 602 that have similar features to that discussed elsewhere herein with respect to the dialysis machine 102 and may include air improvement components 651 and/or potable water supply components 652, having features similar to such components discussed elsewhere herein. Specifically, the air improvements components 651 may purify, cool and/or otherwise improve the air quality in the vicinity of the machine, and the potable water supply components 652 may enable generation and access to potable water at the dialysis machine 602. A display 620 is representationally shown on the screen of the dialysis machine 602 that may include, in various implementations, information corresponding to the dialysis treatment, information concerning the air quality in the vicinity of the dialysis machine 602 and/or information concerning the water quality, i.e. for drinking.

Displayed information, including dialysis treatment information, air quality information, and/or water quality information may also be transmitted via a transmission connection, illustrated as connection 605 which may be a bi-directional communication connection, via a network to a remote computer, including a remote processor, server and/or internet or cloud-based service 630, that may display, analyze and/or generate information 640 concerning the dialysis treatment, air quality and/or water quality reports from the dialysis machine 602.

In an implementation, information from the network (e.g. from a remote computer, server and/or internet or cloud-based service) may be received by the dialysis machine 602 over the connection 605. The received information may include alerts and/or other information concerning general air or water quality, such as air or water quality in a neighborhood of the patient's home, along with instructions concerning the general air or water quality. For example, the instructions may be to close windows if the atmospheric air quality in the area of the patient's home is determined to be poor. In another implementation, the received information may include instructions or commands received from a remote source that may allow scheduling operation and/or triggering of the air improvement components 651 and/or potable water supply components 652 of the dialysis machine 602. For example, certain of the air improvement and potable water supply components may be automatically activated prior to a scheduled dialysis treatment to provide a clean and health-enhanced home environment for the scheduled dialysis treatment. Such information may be provided and exchanged over a secured and encrypted connected health network accessible via components of a cloud-based health service, and reference is made, for example, to US 2018/0316505 A1 to Cohen et al. entitled “Securely Distributing Medical Prescriptions,” which is incorporated herein by reference in its entirety.

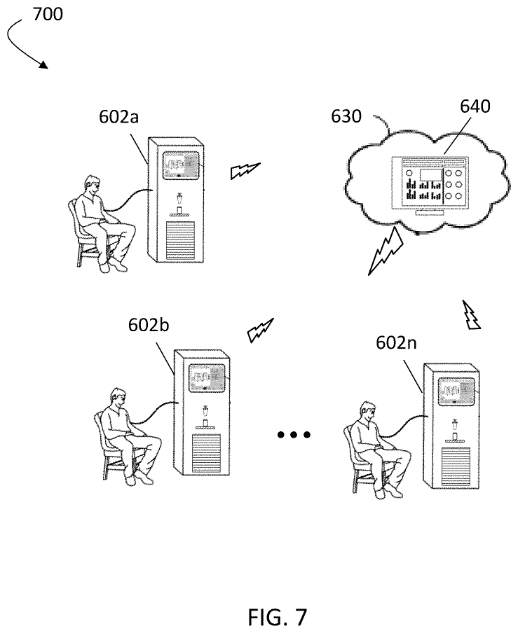

FIG. 7 is a schematic illustration of a connected health network environment 700 exchanging information with multiple home dialysis machines 602a,b,n according to an implementation of the system described herein. In an implementation, the information 640 received at the cloud-based service 630 discussed above may include a collection, compilation and/or anonymized representation of multiple dialysis machines 602a,b,n, each like the dialysis machine 602, that may enable an understanding of the home dialysis environment for multiple households.

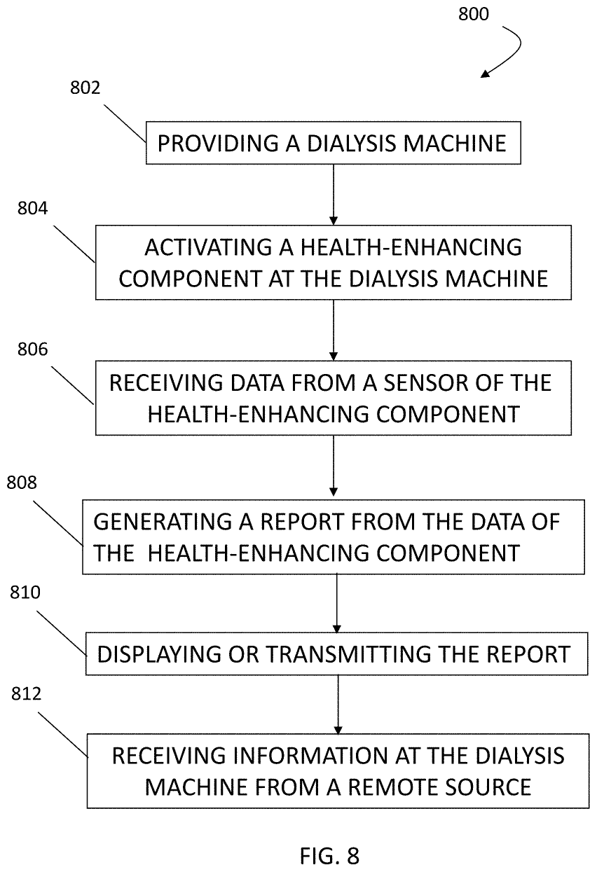

FIG. 8 is a flow diagram 800 of operations for facilitating a health-enhanced home environment using a dialysis machine according to the system described herein (e.g. the dialysis machine 102, 602). At a step 802, a dialysis machine according to the system described herein is provided in a home for performing dialysis treatments on a patient. The dialysis machine may include, integrated therein, one or more health-enhancing components, including an air improvement (e.g. component 151, 651) and a potable water supply component (e.g., component 152, 652). At a step 804, the health-enhancing component is activated at the dialysis machine, for example, to provide purified air and/or potable water. At a step 806, a processor of the dialysis machine receives data from a sensor of the health-enhancing component, such as data corresponding to air and/or water quality. At a step 808, the processor generates a report from the data concerning the health-enhancing component, such as a report on air and/or water quality. It is noted that, in various implementations, the report may be a general information report and/or may be an emergency report that may also act as an alert or alarm in the event of detection of an urgent situation. At a step 810, the report is displayed on a screen of the dialysis machine and/or the report is transmitted over a network to a remote computer. At a step 812, which may optionally be performed, information may be received at the dialysis machine from the remote computer. The information may include for example, information obtained from a database and/or may include an instruction concerning activation of the health-enhancing component and/or information for display on the dialysis machine concerning a health-enhancing feature.

Implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers. Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, a memory card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or similar, should be generally construed to include [a] alone, [b] alone, [c] alone, or any combination of [a], [b] and [c].

Embodiments and implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A dialysis system, comprising:
a dialysis machine;
at least one health-enhancing component integrated into the dialysis machine, wherein the at least one health-enhancing component includes one or more of: an air improvement component that monitors and/or treats air in a vicinity of the dialysis machine, or a potable water supply component that monitors and/or provides potable water from the dialysis machine.

2. The dialysis system of claim 1, wherein the at least one health-enhancing component is the air improvement component.

3. The dialysis system of claim 1, wherein the at least one health-enhancing component is the potable water supply component.

4. The dialysis system of claim 1, wherein the at least one health-enhancing component includes the air improvement component and the potable water supply component.

5. The dialysis system of claim 2, wherein the air improvement component includes one or more of the following: an air purifier, a dehumidifier, an air conditioner, or an air heater.

6. The dialysis system of claim 3, wherein the potable water supply component includes one or more of the following: a water purifier, a minerals supply connection, or a potable water access outlet.

7. The dialysis system of claim 6, wherein the potable water supply component includes the minerals supply connection and a minerals supply cartridge coupled to the minerals supply connection.

8. The dialysis system of claim 1, wherein the at least one health-enhancing component includes a sensor, and wherein the dialysis system further comprises:
at least one processor that receives data from the sensor and generates a report concerning the data.

9. The dialysis system of claim 8, wherein the sensor includes one or more of an air monitor sensor or a water monitor sensor.

10. The dialysis system of claim 8, further comprising:
a remote computer that receives the report over a network from the at least one processor and/or transmits information corresponding to the at least one health-enhancing component to the at least one processor at the dialysis machine.

11. A method for providing a health-enhanced home environment, comprising:
providing a dialysis machine that is capable of delivering a dialysis treatment;
activating at least one health-enhancing component that is integrated into the dialysis machine, wherein the at least one health-enhancing component includes one or more of: an air improvement component that monitors and/or provides treated air in a vicinity of the dialysis machine, or a potable water supply component that monitors or provides potable water from the dialysis machine.

12. The method of claim 11, wherein the at least one health-enhancing component is the air improvement component.

13. The method of claim 11, wherein the at least one health-enhancing component is the potable water supply component.

14. The method of claim 11, wherein the at least one health-enhancing component includes the air improvement component and the potable water supply component.

15. The method of claim 12, wherein the air improvement component includes one or more of the following: an air purifier, a dehumidifier, an air conditioner, or an air heater.

16. The method of claim 13, wherein the potable water supply component includes one or more of the following: a water purifier, a minerals supply connection, or a potable water access outlet.

17. The method of claim 16, wherein the potable water supply component includes the minerals supply connection and a minerals supply cartridge coupled to the minerals supply connection.

18. The method of claim 11, wherein the at least one health-enhancing component includes a sensor, and wherein the method further comprises:
receiving, by at least one processor of the dialysis machine, data from the sensor; and
generating a report concerning the data.

19. The method of claim 18, wherein the sensor includes one or more of an air monitor sensor or a water monitor sensor.

20. The method of claim 18, further comprising:
transmitting, to a remote computer, the report over a network from the at least one processor and/or receiving, from the remote computer, information corresponding to the at least one health-enhancing component.
